(19) United States
(12) Patent Application Publication
(10) Pub. No.: US 2023/0008650 A1
(43) Pub. Date: Jan. 12, 2023

(54) SYSTEMS AND METHODS FOR ASSESSING PATIENT-SPECIFIC RESPONSE TO THROMBOPOIETINRECEPTOR AGONISTS

(71) Applicants: Trustees of Tufts College, Medford, MA (US); University of Pavia, Pavia (IT)

(72) Inventors: Alessandra Balduini, Pavia (IT); David L. Kaplan, Concord, MA (US); Christian A. Di Buduo, Pavia (IT)

(21) Appl. No.: 17/782,989

(22) PCT Filed: Dec. 7, 2020
(86) PCT No.: PCT/US2020/063639

§ 371 Date: Jun. 6, 2022

Related U.S. Application Data
(60) Provisional application No. 62/944,874, filed on Dec. 6, 2019.

(51) Int. Cl. G01N 33/50

(52) U.S. Cl. CPC G01N 33/5073

(57) ABSTRACT

Systems and methods for predicting patient-specific responses to the administration of medicaments that are indicated to modulate megakaryocyte differentiation, proplatelet formation, and/or platelet production are disclosed. The systems and methods can include a three-dimensional bone marrow model that is composed of silk fibroin sponges including a protein of the extracellular matrix, such as fibrinogen. The methods include creating patient-specific megakaryocyte progenitors (or progenitors thereof), seeding those progenitors into the model, introducing the medicament to the progenitors within one model, perfusing the model with a cell culture medium, maturing the progenitors, comparing platelet generation from the model including the medicament to a control model, and generating a report having a prediction of in vivo efficacy based on the comparison.

### CROSS-REFERENCE TO RELATED APPLICATIONS

This application is related to, claims priority to, and incorporates herein for all purposes U.S. Provisional Patent Application No. 62/944,874, filed Dec. 6, 2019.

### STATEMENT REGARDING FEDERALLY FUNDED RESEARCH

This invention was made with government support under grant EB016041 awarded by the National Institutes of Health. The government has certain rights in the invention.

### BACKGROUND

Bone marrow megakaryocytes are responsible for continuous production of platelets in blood, driven by thrombopoietin (TPO) through interaction with its receptor MPL expressed in hematopoietic stem cells, megakaryocyte progenitors, and mature megakaryocytes. In vivo, megakaryocytes associate with bone marrow microvasculature, where they extend proplatelets that protrude through the vascular endothelium into the lumen and release platelets into the bloodstream.

Countless human pathologies result in alterations in platelet production; yet, for many of these, pathogenesis, and thus optimal targeted therapies, remain unknown. A significant advance in treatment of thrombocytopenias is the use of drugs that stimulate platelet production by mimicking the effects of TPO. The TPO-receptor agonists Eltrombopag, Romiplostim, and very recently Avatrombopag, have been approved for treatment of several forms of acquired thrombocytopenia. Inherited Thrombocytopenias are a heterogeneous group of disorders characterized by low platelet count, resulting in impaired hemostasis. While often stable, patients can have hemorrhages and/or excessive bleeding provoked by hemostatic challenges such as trauma or surgery; some hemorrhages appear spontaneously. Treatment of Inherited Thrombocytopenias is still unsatisfactory. For patients affected with the severe forms, that are usually fatal at young ages, the treatment of choice is hematopoietic stem cell transplantation. However, for most patients with Inherited Thrombocytopenias, transplantation is not indicated as the risks outweigh the benefits. In the past, the only effective treatments for these subjects were platelet transfusion to stop or prevent bleeding following trauma or during invasive procedures, anti-fibrinolytic agents, recombinant factor VIIa (rVIIa) or local treatment. However, platelet transfusions expose patients to development of alloimmunization and refractoriness to subsequent platelet infusions, acute reactions, and transmission of infectious diseases. Moreover, the effect of platelet transfusion is transient and short-lived, and long-term administration of platelet concentrates is not feasible due to side effects and shortage of blood donors. Anti-fibrinolytic agents may be effective for mucosa bleeding but create a risk of thrombosis; rVIIa is generally reserved for hemorrhagic emergencies and results have been somewhat equivocal in platelet disorders.

TPO-receptor agonists were first explored in Inherited Thrombocytopenias in 2010 in a phase 2 trial of Eltrombopag in 12 patients with Myosin Heavy Chain 9 (MYH9) mutations. In 2015, Eltrombopag was tested in 8 patients with Wiskott-Aldrich syndrome with platelet increases primarily in the X-linked thrombocytopenia (XLT) patients. More recently, a follow on phase 2 trial showed that Eltrombopag was safe and effective in increasing platelet count and reducing bleeding symptoms in patients with different forms of Inherited Thrombocytopenia, including MYH9-Related Diseases (MYH9-RD), Ankyrin Repeat Domain 26-Related Thrombocytopenia (ANKRD26-RT), XLT/Wiskott-Aldrich syndrome, monoallelic Bernard-Soulier syndrome and Integrin beta 3 (ITGB3)-Related Thrombocytopenia. Further, elective surgeries in MYH9-RD patients with severe thrombocytopenia have been performed safely after administration of Eltrombopag. Overall, these studies indicated that a sizeable proportion of patients with Inherited Thrombocytopenia respond to Eltrombopag, but that the extent of platelet response is highly variable not only among different forms of Inherited Thrombocytopenia, but also among different patients affected by the same disease.

Tools that recapitulate the function of specific tissues or organs are critical to test drug efficacy, reduce ineffective or suboptimal therapies, and personalize the choice of best treatment for each specific patient as exemplified by organoids. Reproduction of the bone marrow has been very difficult because of its incompletely understood complexity. Current research is focused on duplicating characteristic features of the physiologic bone marrow microenvironment ex vivo using relevant biomaterials and bioreactors, along with appropriate human cell sources. Silk is a naturally-derived protein biomaterial with utility for studying platelet production since its fundamental features include non-thrombogenicity, low-immunogenicity, and non-toxicity.

A combination of modular flow chambers and vascular silk tubes and sponges was used to record platelet generation by primary human megakaryocytes, in response to variations in surface stiffness, functionalization with extracellular matrix components, and co-culture with endothelial cells. These systems were able to support efficient platelet formation and, upon perfusion, recovery of functional platelets, as assessed through multiple activation tests, including participation in clot formation and thrombus formation under flow conditions.

A need exists for an ex vivo models of bone marrow that are predictive of in vivo platelet biogenesis and the reaction that such biogenesis has to therapeutics. A need further exists for a personalized medicine approach to this problem, where megakaryocyte progenitors are prepared from a patient's own cells and the resulting ex vivo model of bone marrow provides a personalized prediction of platelet biogenesis response to a therapeutic. In each of these cases, the need requires that the ex vivo models be validated to predict in vivo response.

### SUMMARY

In one aspect, the present disclosure provides a patient-specific method of predicting in vivo efficacy of a Thrombopoietin-receptor agonist for a patient. The method includes: a) preparing from cells acquired from the patient and/or acquiring from the patient-specific haematopoietic stem cells, patient-specific haematopoietic progenitor cells, patient-specific Induced Pluripotent Stem Cells, and/or patient-specific megakaryocyte progenitors; b) seeding the patient-specific haematopoietic stem cells, the patient-specific haematopoietic progenitor cells, the patient-specific Induced Pluripotent Stem Cells, and/or the patient-specific megakaryocyte progenitors into at least two three-dimensional bone marrow models, each of the at least two three-dimensional bone marrow models comprising a regenerated silk fibroin sponge having an interconnected network of pores with pore sizes and pore distributions adapted to facilitate cell adhesion, proplatelet extension, proplatelet formation, and platelet release and collection from maturing haematopoietic stem cells, haematopoietic progenitor cells, Induced Pluripotent Stem Cells, and/or megakaryocyte progenitors seeded therein, the at least two three-dimensional bone marrow models including an experimental model and a control model, each of the regenerated silk fibroin sponges including at least one protein of the extracellular matrix; c) introducing the medicament to the patient-specific haematopoietic stem cells, the patient-specific haematopoietic progenitor cells, the patient-specific Induced Pluripotent Stem Cells, and/or the patient-specific megakaryocyte progenitors within the experimental model; d) perfusing the at least two three-dimensional bone marrow models with a cell culture medium; e) maturing the patient-specific haematopoietic stem cells, the patient-specific haematopoietic progenitor cells, the patient-specific Induced Pluripotent Stem Cells, and/or the patient-specific megakaryocyte progenitors into patient-specific megakaryocytes, including cell adhesion, proplatelet extension, proplatelet formation, and platelet release and collection; f) comparing platelet production from the patient-specific megakaryocytes within the experimental model versus the patient-specific megakaryocytes within the control model, the control model lacking the medicament; and g) generating a report, wherein the report includes a prediction of in vivo platelet production of the patient upon having the medicament administered, the prediction incorporating the comparing of step f), wherein the prediction is validated to have a statistically significant correlation between an increase in platelet count ex vivo and in vivo, expressed either as fold increase or absolute platelet numbers, with an R-squared value of at least 0.6 and a p value of less than 0.001 in a population study having at least 8 patients.

In another aspect, the present disclosure provides a patient-specific method of administering a medicament to a patient in need thereof. The medicament is indicated to modulate megakaryocyte differentiation, proplatelet formation, and/or platelet production in the patient. The method includes: a) preparing from cells acquired from the patient and/or acquiring from the patient patient-specific haematopoietic stem cells, patient-specific haematopoietic progenitor cells, patient-specific Induced Pluripotent Stem Cells, and/or patient-specific megakaryocyte progenitors; b) seeding the patient-specific haematopoietic stem cells, the patient-specific haematopoietic progenitor cells, the patient-specific Induced Pluripotent Stem Cells, and/or the patient-specific megakaryocyte progenitors into at least two three-dimensional bone marrow models, each of the at least two three-dimensional bone marrow models comprising a regenerated silk fibroin sponge having an interconnected network of pores with pore sizes and pore distributions adapted to facilitate cell adhesion, proplatelet extension, proplatelet formation, and platelet release and collection from maturing haematopoietic stem cells, haematopoietic progenitor cells, Induced Pluripotent Stem Cells, and/or megakaryocyte progenitors seeded therein, the at least two three-dimensional bone marrow models including an experimental model and a control model, each of the regenerated silk fibroin sponges including at least one protein of the extracellular matrix; c) introducing the medicament to the patient-specific haematopoietic stem cells, the patient-specific haematopoietic progenitor cells, the patient-specific Induced Pluripotent Stem Cells, and/or the patient-specific megakaryocyte progenitors within the experimental model; d) perfusing the at least two three-dimensional bone marrow models with a cell culture medium; e) maturing the patient-specific haematopoietic stem cells, the patient-specific haematopoietic progenitor cells, the patient-specific Induced Pluripotent Stem Cells, and/or the patient-specific megakaryocyte progenitors into patient-specific megakaryocytes, including cell adhesion, proplatelet extension, proplatelet formation, and platelet release and collection; f) comparing platelet production from the patient-specific megakaryocytes within the experimental model versus the patient-specific megakaryocytes within the control model, the control model lacking the medicament; g) generating a report, wherein the report includes a prediction of in vivo platelet production of the patient upon having the medicament administered, the prediction incorporating the comparing of step f), wherein the prediction is validated to have a statistically significant correlation between an increase in platelet count ex vivo and in vivo, expressed either as fold increase or absolute platelet numbers, with an R-squared value of at least 0.6 and a p value of less than 0.001 in a population study having at least 8 patients; and h) subsequent to step g), administering medicament to the patient if the prediction exceeds a predetermined threshold and foregoing administering the medicament to the patient if the prediction fails to exceed the predetermined threshold.

### BRIEF DESCRIPTIONS OF THE DRAWINGS

FIG. 1 shows a silk sponge bone marrow perfusion system and some corresponding images and data. (A-C) A peristaltic pump drives perfusion of the cell culture medium from a reservoir to the device equipped with a silk fibroin sponge prepared directly inside the chamber by dispensing an aqueous silk solution mixed with salt particles (scale bar B=1.5 cm; scale bar C=2 mm). After leaching out the salt, the resulting porous silk sponge can be sterilized. (D,E) Confocal microscopy reconstruction of the silk sponge showed the presence of an interconnected alveolar network (scale bar D=200 μm; scale bar E=200 μm). (F) The analysis of pore diameters measured on top and bottom of the scaffold demonstrated no significant differences throughout the scaffold. Results are presented as mean±SD (n=150 pore/condition, p=NS). (G) Confocal microscopy analysis of CFSE⁺ cells cultured within the silk scaffold (highlighted=CFSE; grey=silk; scale bar=50 μm).

FIG. 2 shows workflow and data associated with modeling physiological and pathological megakaryopoiesis. (A) Megakaryocytes were differentiated from healthy controls and patients affected by MYH9-RD and ANKRD26-RT patients and cultured into the bone marrow device in presence of 10 ng/mL TPO. (B) Output of CD41⁺CD42b⁺megakaryocyte at the end of differentiation relative to healthy controls (n=12 Healthy Controls, n=12 1VIYH9-RD; n=12 ANKRD26-RT; p=NS) (C) Percentage of proplatelet formation relative to healthy controls (n=12 Healthy Controls, n=12 MYH9-RD; n=12 ANKRD26-RT; *p<0.01). (D) Number of proplatelet bifurcation per single megakaryocytes in healthy controls and patients (n=12 Healthy Controls, n=12 MYH9-RD; n=12 ANKRD26-RT; *p<0.01). (E) Representative immunofluorescence staining of proplatelet structure (scale bar=20 μm). Decreased proplatelet branching can be observed in patients with respect to heathy controls. All results are presented as mean±SD.

FIG. 3 illustrates evidence of Eltrombopag promoting megakaryocyte differentiation ex vivo. (A) Megakaryocytes were differentiated from peripheral blood progenitors of patients affected by MYH9-RD or ANKRD26-RT and cultured in the silk bone marrow tissue device in the presence of 10 ng/mL TPO supplemented or not with 500 ng/mL Eltrombopag (EPAG) and analyzed. (B) Representative immunofluorescence staining of CD61 (scale bar=25 μm) and (C) analysis of ploidy levels at the end of the culture (TPO: n=3 MYH9-RD; n=3 ANKRD26-RT; TPO+EPAG: n=3 MYH9-RD; n=3 ANKRD26-RT; p=NS). (D) Representative flow cytometry analysis of CD41⁺CD42b⁺megakaryocytes at the end of the culture and (E) statistical analysis of mean fluorescence intensity (MFI) of the markers (TPO: n=12 MYH9-RD; n=12 ANKRD26-RT; TPO+EPAG: n=12 MYH9-RD; n=12 ANKRD26-RT; p=NS). (F) Output was calculated as the fold increase in the percentage of CD41⁺CD42b⁺ cells in presence of TPO+EPAG with respect to the percentage of double positive cells in presence of TPO alone (ALL: n=24 thrombocytopenic patients; ANKRD26-RT: n=12; MYH9-RD: n=12, p<0.05). All results are presented as mean±SD.

FIG. 4 illustrates evidence of Eltrombopag sustaining increased proplatelet formation ex vivo. (A) Confocal microscopy analysis of 3D megakaryocyte culture imaged at the end of differentiation. Megakaryocytes were elongating proplatelet shafts, which assemble nascent platelets at their terminal ends, within the hollow space of silk pores (scale bars=50 μm). (Av-viii) Analysis of proplatelet structure was performed by immunofluorescence staining of the megakaryocyte-specific cytoskeleton component β1-tubulin (scale bar=25 μm). In both diseases, the representative pictures show increased elongation and branching of proplatelet shafts in presence of TPO+EPAG with respect to TPO alone. (B) The percentage of proplatelet forming megakaryocytes was calculated as the number of cells displaying long filamentous pseudopods with respect to total number of round megakaryocytes per analyzed field (TPO: n=121VIYH9-RD; n=12 ANKRD26-RT; TPO+EPAG: n=12 MYH9-RD; n=12 ANKRD26-RT; **p<0.01; *p<0.05). (C) Graphs show the fold increase of proplatelet formation in presence of TPO+EPAG with respect to TPO alone (TPO: n=12 MYH9-RD; n=12 ANKRD26-RT; TPO+EPAG: n=12 IVIYH9-RD; n=12 ANKRD26-RT; p<0.05). All results are presented as mean±SD.

FIG. 5 illustrates evidence that ex vivo platelet count is predictive of response to treatments. (A) The flow chamber was perfused with culture media and released platelets collected into gas-permeable bags before counting by flow cytometry. (B) Light microscopy and immunofluorescent analysis of the collected medium demonstrated presence of large pre-platelets, dumbbells and little discoid platelets having the microtubule coil typically present in resting platelets (scale bars=10 μm). (C) Representative flow cytometry analysis of expression of CD41 and CD42b surface markers. (D) Platelet count was calculated with counting beads for ex vivo treated samples. The fold increase was calculated as the ratio between platelet count in the presence of TPO+EPAG with respect to TPO alone. Results are presented as mean±SD (TPO: n=81VIYH9-RD; n=9 ANKRD26-RT; TPO+EPAG: n=8 MYH9-RD; n=9 ANKRD26-RT; p<0.05). (E) Analysis of the correlation between the fold increase in ex vivo platelet production and the increase of platelet count observed in vivo from the same patient (n=8 MYH9-RD; n=9 ANKRD26-RT; p<0.0001).

### DETAILED DESCRIPTION

Before the present invention is described in further detail, it is to be understood that the invention is not limited to the particular embodiments described. It is also understood that the terminology used herein is for the purpose of describing particular embodiments only, and is not intended to be limiting. The scope of the present invention will be limited only by the claims. As used herein, the singular forms “a”, “an”, and “the” include plural embodiments unless the context clearly dictates otherwise.

Specific structures, devices and methods relating to conducting ex vivo predictions of in vivo medicament efficacy are disclosed. It should be apparent to those skilled in the art that many additional modifications beside those already described are possible without departing from the inventive concepts. In interpreting this disclosure, all terms should be interpreted in the broadest possible manner consistent with the context. Variations of the term “comprising” should be interpreted as referring to elements, components, or steps in a non-exclusive manner, so the referenced elements, components, or steps may be combined with other elements, components, or steps that are not expressly referenced. Embodiments referenced as “comprising” certain elements are also contemplated as “consisting essentially of” and “consisting of” those elements. When two or more ranges for a particular value are recited, this disclosure contemplates all combinations of the upper and lower bounds of those ranges that are not explicitly recited. For example, recitation of a value of between 1 and 10 or between 2 and 9 also contemplates a value of between 1 and 9 or between 2 and 10.

As used herein, “three-dimensional” refers to a culture condition where haematopoietic progenitors are seeded within a silk-based 3D scaffold housing as little as 10⁵ cells/scaffold, that can be perfused to collect and count platelets.

As used herein, “two-dimensional” refers to the seeding of haematopoietic progenitors within a petri dish or flask housing cells in a static liquid culture that cannot be perfused and does not allow platelet collection.

The present disclosure provides methods of ex vivo determination of in vivo efficacy of medicaments, including Thrombopoietin-receptor agonists and other treatments intended to modulate megakaryocyte differentiation, proplatelet formation, and/or platelet production. In one case, the methods provide a prediction of efficacy. In some cases, the methods provide administering the medicament with a dosing strategy that is informed by the prediction. In some cases, where the ex vivo model determines that platelet production in the patient is unlikely, the methods can include foregoing administering the treatment with the medicament.

In one aspect, the present disclosure provides a patient-specific method of predicting in vivo efficacy of a medicament. The medicament is a medicament indicated to modulate megakaryocyte differentiation, proplatelet formation, and/or platelet production in a patient. The medicament can be a Thrombopoietin-receptor agonist. The method includes: a) preparing from cells acquired from the patient and/or acquiring from the patient patient-specific haematopoietic stem cells, patient-specific haematopoietic progenitor cells, patient-specific Induced Pluripotent Stem Cells, and/or patient-specific megakaryocyte progenitors; b) seeding the patient-specific haomatopoietic stem cells, the patient-specific haematopoietic progenitor cells, the patient-specific Induced Pluripotent Stem Cells, and/or the patient-specific megakaryocyte progenitors into at least two three-dimensional bone marrow models, each of the at least two three-dimensional bone marrow models comprising a regenerated silk fibroin sponge having an interconnected network of pores with pore sizes and pore distributions adapted to facilitate cell adhesion, proplatelet extension, proplatelet formation, and platelet release and collection from maturing haematopoietic stem cells, haematopoietic progenitor cells, Induced Pluripotent Stem Cells, and/or megakaryocyte progenitors seeded therein, the at least two three-dimensional bone marrow models including an experimental model and a control model, each of the regenerated silk fibroin sponges including at least one protein of the extracellular matrix; c) introducing the medicament to the patient-specific haematopoietic stem cells, the patient-specific haematopoietic progenitor cells, the patient-specific Induced Pluripotent Stem Cells, and/or the patient-specific megakaryocyte progenitors within the experimental model; d) perfusing the at least two three-dimensional bone marrow models with a cell culture medium; e) maturing the patient-specific haematopoietic stem cells, the patient-specific haematopoietic progenitor cells, the patient-specific Induced Pluripotent Stem Cells, and/or the patient-specific megakaryocyte progenitors into patient-specific megakaryocytes, including cell adhesion, proplatelet extension, proplatelet formation, and platelet release and collection; f) comparing platelet production from the patient-specific megakaryocytes within the experimental model versus the patient-specific megakaryocytes within the control model, the control model lacking the medicament; and g) generating a report, wherein the report includes a prediction of in vivo platelet production of the patient upon having the medicament administered, the prediction incorporating the comparing of step f), wherein the prediction is validated to have a statistically significant correlation between an increase in platelet count ex vivo and in vivo, expressed either as fold increase or absolute platelet numbers, with an R-squared value of at least 0.6 and a p value of less than 0.001 in a population study having at least 8 patients.

In another aspect, the present disclosure provides a method of administering a medicament to a patient in need thereof. The method includes steps a), b), c), d), e), f), and g) of the method described in the immediately preceding paragraph. The method further includes: h) subsequent to step g), administering the medicament to the patient if the prediction exceeds a predetermined threshold and foregoing administering the medicament to the patient if the prediction fails to exceed the predetermined threshold.

Either of these methods can further include analyzing megakaryocyte differentiation of the patient-specific megakaryocytes. This analyzing can provide further information into the therapeutic effect of the administered agonist. The report can include the outcome of this analyzing.

Without wishing to be bound by any particular theory, it is believed that the correlation between the impact of a medicament on platelet generation (and megakaryocyte differentiation) of the ex vivo models to in vivo efficacy is highly unpredictable before the model has been validated for the first time ever to confirm this correlation. As a result, simply the suggestion of constructing such a model and making predictions is not adequate to lead a person having ordinary skill in the art to have a reasonable expectation of success. In other words, if a person having ordinary skill in the art were to attempt to construct such an ex vivo model for a randomly-selected condition, the chances of success in predicting in vivo efficacy with that ex vivo model would have been inherently low prior to the present invention, because no such models had ever been validated previously. The details of the present disclosure are tightly hewn to the validated capabilities of the models described herein.

The statistically significant correlation between ex vivo platelet production and in vivo platelet production can have an R-squared value of at least 0.6 or at least 0.70 and a p value of less than 0.001 or less than 0.0001 in a population study having at least 8 patients.

Preparing patient-specific megakaryocyte progenitors can be done by methods known to those having ordinary skill in the art, including but not limited to, harvesting a sample from a patient (e.g., a blood sample), generating Induced Pluripotent Stem Cells (iPSCs) from the sample, and differentiating the iPSCs into differentiated megakaryocytes.

In some cases, the three-dimensional bone marrow models described herein can comprise, consist essentially of, or consist of a fibronectin-functionalized silk fibroin foam made from regenerated silk fibroin. In some cases, the three-dimensional bone marrow models described herein can be substantially free of tubular blood vessel mimicking structures, such as the silk fibroin tubes utilized in some models. In some cases, the interconnected network of pores within the three-dimensional bone marrow models can have substantially uniform pore sizes.

The at least one protein of the extracellular matrix can be selected from the group consisting of a proteoglycan, hyaluronic acid, a collagen, elastin, fibronectin, fibrin, fibrinogen, a laminin, thrombospondin, and combinations thereof. In some cases, the at least one protein of the extracellular matrix is fibronectin.

The medicament can be selected from the group consisting of Thrombopoietin-receptor agonists, which can be selected from the group consisting of Eltrombopag, Romiplostim, Avatraombopag, and other Thrombopoietin-receptor agonists known to those having ordinary skill in the art, or from any other treatment intended to modulate megakaryocyte differentiation, proplatelet formation and/or platelet production, including Rho kinase inhibitors (ROCK), MAP kinase inhibitor, modulators of the WNT pathway and/or antagonists of the aryl hydrocarbon receptor.

The condition being treated by the agonists described herein (and the efficacy of which the models herein predict) can be Thrombocytopenic disorders, such as Inherited Thrombocytopenias or Immunothrombocytopenia (ITP).

In some cases, the patient has a particular gene variant, such as ANKRD26-RT or MYH9-RD.

### Example 1

### Materials

B. mori silkworm cocoons were supplied by Tajima Shoji Co., Ltd. (Yokohama, Japan). Pharmed tubing was from Cole-Parmer (Vernon Hills, Ill., USA). Immunomagnetic separation system was from Miltenyi Biotech (Bergisch Gladbach, Germany and Bologna, Italy). Recombinant human thrombopoietin (TPO), interleukin-6 (IL-6), interleukin-11 (IL-11)were from Peprotech (London, UK). TruCount tubes and human fibronectin were from Becton Dickinson (S. Jose, CA, USA). The following antibodies were used: mouse monoclonal anti-CD61, clone SZ21, from Immunotech (Marseille, France); rabbit monoclonal anti-β1-tubulin from Abcam. Alexa Fluor-conjugated secondary antibodies and Hoechst 33258 were from Life Technologies (Monza, Italy).

### Production of the Drug-Testing Device

The chamber was manufactured using 3D FDM printing technology and biocompatible silicon molding approach. The modelling of the bioreactor was created using a CAD software and used to generate 3D negative mold components exported as STL (Standard Triangulation Language) files, sliced with Slic3R PE and export to the 3D printer. The printing is done using a poly(lactic acid) (PLA) high-temperature filament of 1.75 mm (FormFutura, Netherland) deployed in layers of 100 μm by a 0.25 mm nozzle. After printing, the mold was cured in an oven at 100° C. for 20 min to increase mechanical properties. To produce the perfusion channel, 21G needles were disposed in the dedicate holes and sealed with a gel of 25% Pluronic F-127. The molding was performed using a polydimethylsiloxane (PDMS) (Sylgard®184, Dow Corning), mixed in a 10:1 ratio of base material and curing agent. The selected material is stable both at low and high temperatures (45° C. to 200° C.) and it is resistant to UV, water, and solvents. The PDMS was poured into the 3D printed molds that were positioned into a vacuum chamber to remove all the air bubbles. The curing of the PDMS was performed in a dried oven 70° C. for 4 hours; the molds were then dissociated of the final silicon models sterilizable by autoclave. The chamber consisted of two wells of 22x10 mm, having a hollow cavity of 15x2 mm enclosed in a block of 30×30 mm and connected to the outside of the chamber through channels of 0.9 mm diameter. The luer adaptors for the inlet and outlet were mounted in the channel and sealed with biocompatible silicone adhesive MD7-4502 (Dow Corning, USA). Then, the modular flow chamber was equipped with a silk fibroin sponge functionalized with fibronectin as described subsequently.

### Preparation of the Silk Fibroin Solution

Silk fibroin aqueous solution was obtained from B. mori silkworm cocoons according to previously published literature (Di Buduo, C. A., Abbonante, V., Tozzi, L., Kaplan, D. L., & Balduini, A. (2018). Three-Dimensional Tissue Models for Studying Ex vivo Megakaryocytopoiesis and Platelet Production. Methods Mol Biol, 1812, 177-193). Briefly, dewormed cocoons were boiled for 30 min in 0.02 M Na2CO3 solution at a weight to volume ratio of 10 g to 4 L. The fibers were rinsed for 20 min for three times in ultrapure water and dried overnight. The dried fibers were solubilized for 4 h at 60° C. in 9.3 M LiBr at a weight to volume ratio of 3 g/12 mL. The solubilized silk solution was dialyzed against distilled water using a Slide-A-Lyzer cassette (Thermo Scientific, Waltham, Mass., USA) with a 3500 MW cutoff for three days and changing the water a total of eight times. The silk solution was centrifuged at maximum speed for 15 min to remove large particulates and stored at 4° C. The concentration of the silk solution was determined by drying a known volume of the solution overnight at 60° C. and massing the remaining solids.

### Silk Bone Marrow Fabrication and Assembly

Silk solution (8% w/v) (Lovett, M., Cannizzaro, C., Daheron, L., Messmer, B., Vunjak-Novakovic, G., & Kaplan, D. L. (2007). Silk fibroin microtubes for blood vessel engineering. Biomaterials, 28(35), 5271-5279.) was mixed with 25 μg/mL fibronectin and dispensed into the modular chamber. NaCl particles (approximately 500 μm in diameter) were then sifted into the solution in a ratio of 1 mL to 2 g of NaCl particles as porogen. The scaffolds were then placed at room temperature for 48 hours and then soaked in distilled water for 48 hours to leach out the NaCl particles. The scaffolds were sterilized in 70% ethanol and finally rinsed five times in PBS over 24 hours. Silk scaffolds were characterized by confocal, scanning electron microscopy, as subsequently described. Perfusion of the silk scaffold has been tested at different flow rates (1-50 μL/min) by using a peristaltic pump. The total volume collected after each test corresponded to that injected in the system by the pump.

### Patients

Human peripheral blood samples were obtained from healthy controls and thrombocytopenic patients after informed consent. All samples were processed following the ethical committee of the I.R.C.C. S. Policlinico San Matteo Foundation and the principles of the Helsinki Declaration. Diagnosis of MYH9-RD or ANKRD26-RT had been confirmed by genetic analysis in all the cases. All patients provided written informed consent for this study, which was approved by the Institutional Review Board of the IRCCS Policlinico San Matteo Foundation, Pavia, Italy. A sample of 15 mL of peripheral venous blood anticoagulated with ACD was collected for the analysis in the 3D bone marrow system. Some patients had previously received a short-term course of Eltrombopag either within a phase 2 clinical trial (Zaninetti, C., Gresele, P., Bertomoro, A., Klersy, C., De Candia, E., Veneri, D., Barozzi, S., Fierro, T., Alberelli, M. A., Musella, V., Noris, P., Fabris, F., Balduini, C. L., & Pecci, A. (2019). Eltrombopag for the treatment of inherited thrombocytopenias: a phase 2 clinical trial. Haematologica) or in preparation for elective surgery (Zaninetti, C., Barozzi, S., Bozzi, V., Gresele, P., Balduini, C. L., & Pecci, A. (2019). Eltrombopag in preparation for surgery in patients with severe MYH9-related thrombocytopenia. Am J Hematol, 94(8), E199-E201). In any case, Eltrombopag was given at the dose of 50 or 75 mg/day for 3 or 6 weeks (Zaninetti, Barozzi, et al., 2019; Zaninetti, Gresele, et al., 2019). The in vivo clinical response to the drug was expressed as the absolute increase in platelet count at the end of Eltrombopag treatment with respect to baseline. Blood samples for this study were collected when patients were out of Eltrombopag therapy and had platelet count at their baseline levels.

### Human Megakaryocytes Differentiation Within the Silk Bone Marrow

CD45⁺hematopoietic stem cells from peripheral blood samples were separated by immunomagnetic bead selection kit (Miltenyi Biotec, Bologna, Italy) and cultured for 6 days in flask in presence in Stem Span media (StemCell Technologies, Canada) supplemented with 1% penicillin-streptomycin, 1% L-glutamine, 10 ng/mL TPO, IL-6 and IL-11 in presence or not of 500 ng/mL Eltrombopag (Novartis) at 37° C. in a 5% CO2 fully humidified atmosphere, as previously described (Bluteau, D., Balduini, A., Balayn, N., Currao, M., Nurden, P., Deswarte, C., Leverger, G., Noris, P., Perrotta, S., Solary, E., Vainchenker, W., Debili, N., Favier, R., & Raslova, H. (2014). Thrombocytopenia-associated mutations in the ANKRD26 regulatory region induce MAPK hyperactivation. J Clin Invest, 124(2), 580-591; and Pecci, A., Malara, A., Badalucco, S., Bozzi, V., Torti, M., Balduini, C. L., & Balduini, A. (2009)). Megakaryocytes of patients with MYH9-related thrombocytopenia present an altered proplatelet formation. Thromb Haemost, 102(1), 90-96).

At day 6, CD61⁺early megakaryocytic progenitors were sorted by immunomagnetic selection kit (Miltenyi Biotec, Bologna, Italy) and seeded for additional 8 days within the silk bone marrow model in presence of 10 ng/mL TPO supplemented or not with 500 ng/mL Eltrombopag.

At day 14 of differentiation, the chamber was sealed, and the outlet ports were connected to the outlet needles. Culture media-filled tubes were connected to the inlet needles. The chamber was placed into the incubator (37° C. and 5% CO2), and transfer bags for platelet collection were secured to the outlet ports. The peristaltic pump was placed outside the incubator and media was pumped for 4 hours at a flow rate of 10 μL/min, speed range: 0.18 rpm, perfusion pause: 120 sec, perfusion run: 5 min with a peristaltic pump.

Evaluation of Differentiation and Proplatelet Formation by ex vivo Differentiated Megakaryocytes

Megakaryocyte differentiation and proplatelet yields were evaluated by adhesion on fibronectin at the end of the culture (14^{th} day), as previously described (Di Buduo, C. A., Moccia, F., Battiston, M., De Marco, L., Mazzucato, M., Moratti, R., Tanzi, F., & Balduini, A. (2014). The importance of calcium in the regulation of megakaryocyte function. Haematologica, 99(4), 769-778; and Pecci et al., 2009). Briefly, 12 mm glass cover-slips were coated with 25 μg/ml human fibronectin (Merck-Millipore, Milan, Italy), for 24 hours at 4° C. Megakaryocytes were harvested from the silk bone marrow scaffold by extensive washing and seeded in a 24-well plate, at 37° C. in a 5% CO2 fully humidified atmosphere. After 16 hours, adhering cells were fixed in 4% paraformaldehyde (PFA), permeabilized with 0.1% Triton X-100 (Sigma Aldrich, Milan, Italy), and stained for immunofluorescence evaluation with rabbit anti-β1-tubulin primary antibody (1:700) or anti-mouse CD61 (1:100) and Alexa Fluor-conjugated secondary antibodies (1:500) (Invitrogen, Milan, Italy). Nuclei were stained with Hoechst 33258 (1:10,000) (Sigma Aldrich, Milan, Italy). The cover-slips were mounted onto glass slides with ProLong Gold antifade reagent (Invitrogen, Milan, Italy) and imaged by an Olympus BX51 microscope (Olympus, Deutschland GmbH, Hamburg, Germany). Proplatelet-forming megakaryocytes were identified as cells displaying long filamentous structure ending with platelet-sized tips. The results were expressed as a percentage of the total number of cells analyzed.

### Imaging of Megakaryocyte Cultures Within the 3D Silk Bone Marrow Model

For immunofluorescence imaging of megakaryocyte cultures within the silk bone marrow tissue model, samples were fixed in 4% paraformaldehyde (PFA) for 20 minutes and then blocked with 5% bovine serum albumin (BSA, Sigma) for 30 minutes at room temperature. Samples were probed with anti-CD61 (1:100) overnight at 4° C., and then immersed in Alexa Fluor secondary antibody (1:500) for 2 hours at room temperature. Nuclei were stained with Hoechst. Samples were imaged by a TCS SP8 confocal laser scanning microscope (Leica, Heidelberg, Germany). For silk fibroin scaffolds imaging, we took advantage of silk auto-fluorescence in UV light. In some experiments, silk fluorescence was brightened by staining with Hoechst (Talukdar, S., Nguyen, Q. T., Chen, A. C., Sah, R. L., & Kundu, S. C. (2011). Effect of initial cell seeding density on 3D-engineered silk fibroin scaffolds for articular cartilage tissue engineering. Biomaterials, 32(34), 8927-8937). For all immunofluorescence imaging, the acquisition parameters were set on the negative controls. 3D reconstruction and image processing performed using Leica licensed software or Image J software.

### Evaluation of Platelet Morphology

For analysis of peripheral blood and ex vivo collected platelet morphology, different approaches were used. First, megakaryocytes at the end of differentiation and platelets from peripheral blood or perfused media were visualized by light microscopy with an Olympus IX53 (Olympus Deutschland GmbH, Hamburg, Germany). For analysis of cytoskeleton components, cells were stained as previously described (Di Buduo, C. A., Alberelli, M. A., Glembostky, A. C., Podda, G., Lev, P. R., Cattaneo, M., Landolfi, R., Heller, P. G., Balduini, A., & De Candia, E. (2016). Abnormal proplatelet formation and emperipolesis in cultured human megakaryocytes from gray platelet syndrome patients. Sci Rep, 6, 23213). Briefly, collected platelets were fixed in 4% PFA and centrifuged onto poly-L-lysine coated coverslip while peripheral blood smears were air-dried and then fixed in 4% PFA, permeabilized with 0.1% Triton X-100 for 5 minutes and blocked with 5% BSA for 30 minutes at room temperature. In order to visualize microtubule organization, samples were probed with anti-β1-tubulin (1:1000) for 1 hour at room temperature and then immersed in Alexa Fluor secondary antibody (1:500) for 2 hours at room temperature. Samples were mounted onto glass slides with ProLong Gold antifade reagent (Invitrogen, Milan, Italy) and then imaged by an Olympus BX51 fluorescence microscope (Olympus, Deutschland GmbH, Hamburg, Germany). For all immunofluorescence imaging, the acquisition parameters were set on the negative controls, which were routinely performed by omitting the primary antibody.

### Flow Cytometry

Flow cytometry settings for analysis of megakaryocytes and ex vivo generated platelets were established, as previously described (Abbonante, V., Di Buduo, C. A., Gruppi, C., Malara, A., Gianelli, U., Celesti, G., Anselmo, A., Laghi, L., Vercellino, M., Visai, L., Iurlo, A., Moratti, R., Barosi, G., Rosti, V., & Balduini, A. (2016). Thrombopoietin/TGF-(31 Loop Regulates Megakaryocyte Extracellular Matrix Component Synthesis. Stem Cells, 34(4), 1123-1133). For analysis of the percentage of fully differentiated megakaryocytes at the end of the culture (14^{th} day), 50×10³ cells were suspended in phosphate buffer saline (PBS) and stained with a FITC-conjugated antibody against human CD41 and human CD42b (PE) (eBioscience, Milan, Italy) at room temperature in the dark for 30 minutes and then analyzed. Ex vivo collected platelets were analyzed using the same forward, and side scatter pattern as human peripheral blood and identified as CD41⁺CD42b⁺events. Isotype controls were used as negative controls to exclude non-specific background signal. The platelet number was calculated using a TruCount bead standard. The ex vivo response to the drug was expressed either as the absolute increase or the fold increase in platelet count at the end of the culture with TPO+EPAG with respect to TPO alone. A minimum of 10.000 events were acquired. All samples were acquired with a Beckman Coulter Navios flow cytometer (Indianapolis, Ind., US). Off-line data analysis was performed using Beckman Coulter Navios software package.

### Statistics

Values were expressed as mean plus or minus the standard deviation (mean±SD). Two-tailed Student's t-test was performed for statistical analysis. Statistical analysis was performed with GraphPad Software. A p-value less than 0.05 was considered statistically significant. All experiments were independently replicated at least three times.

### Device Design and Prototyping

In adults, hematopoietic bone marrow is located in the medullary cavity of flat and long bones (Travlos, G. S. (2006). Normal structure, function, and histology of the bone marrow. Toxicol Pathol, 34(5), 548-565), served by blood vessels that branch out into millions of small thin-walled arterioles and sinusoids allowing mature blood cells to enter the bloodstream. To mimic such a structure, a device prototype of rectangular shape with 30×30 mm size and hollow cavities of 15x2 mm was developed. The device was connected to an outside peristaltic electronic pump through 0.9 mm diameter channels equipped with luer lock adaptors. We used devices with up to 2 reservoirs; however, the chamber can be designed to provide as many channels as required by the experimental conditions. Crosstalk between channels inside the device was eliminated by appropriate spatial separation and independent perfusion to allow assessment of patient-specific responses, following simultaneous exposure to TPO alone and TPO in combination with the tested drug.

3D printing technology is one emerging option for producing new devices at a customized, fast and cost-effective manner. The printing process for the negative mold of our device is easily scalable. It can be created in less than 1 hour using a polylactic acid, which allows casting and curing of polydimethylsiloxane (PDMS), a non-toxic polymeric organosilicon. The final shape of the system is optically clear. Importantly, the device is reusable and autoclavable in order to ensure overall sterility to the system.

### Silk Biomaterials for Bone Marrow System Assembly and Characterization

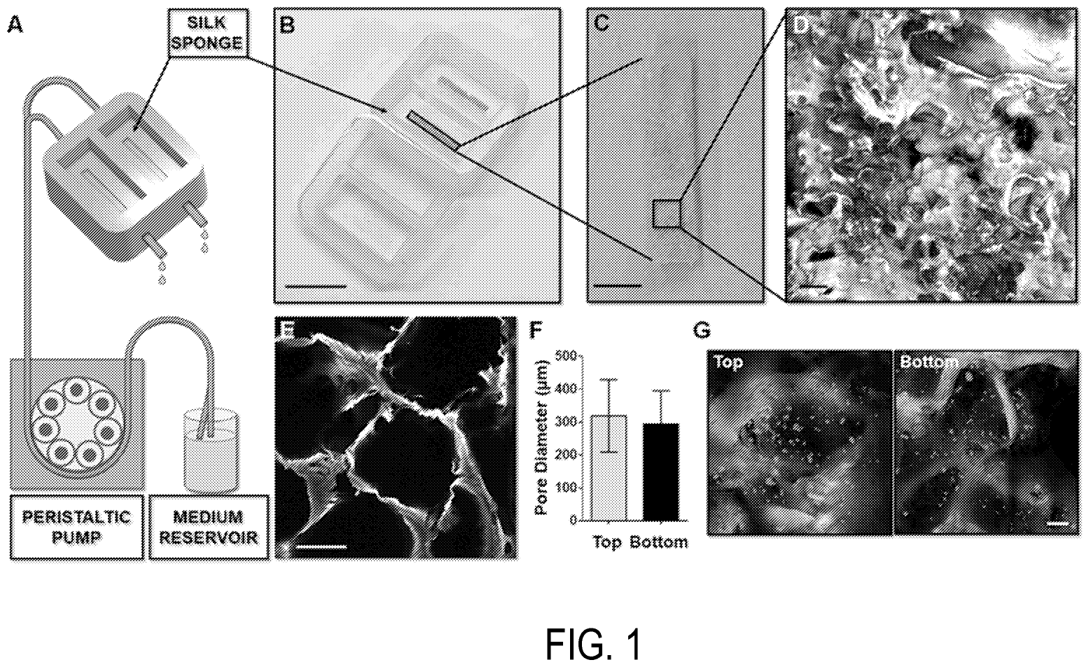

A silk fibroin structure functionalized with fibronectin was prepared with salt leaching method and inserted into the device to model a spongy scaffold that reproduces bone marrow architecture, composition and microcirculation (FIG. 1A-C). A 2-days production process allowed to obtain a sterile 3D silk-fibronectin scaffold that could be stored in water, at 4° C., up to one month after preparation and used upon experimental needs. The silk scaffold was connected to gas-permeable tubing allowing perfusion of the media with a peristaltic pump connected to inlet and outlet ports (FIG. 1A). A cover cap closes the system before starting perfusion. The 3D reconstruction of the silk scaffold revealed the presence of multiple spatially-distinct niches (FIGS. 1D and 1E) and also demonstrated homogeneous distribution of pores from top to bottom of the scaffold (FIG. 1F). This arrangement efficiently supported diffusion of cells (FIG. 1G) and media outflow without altering the shape and integrity of the silk. Importantly, the total volume collected after perfusion corresponded to that injected in the system by the pump.

Tuning of the Silk Bone Marrow Device for Testing Hematopoietic Progenitor Response to Drugs

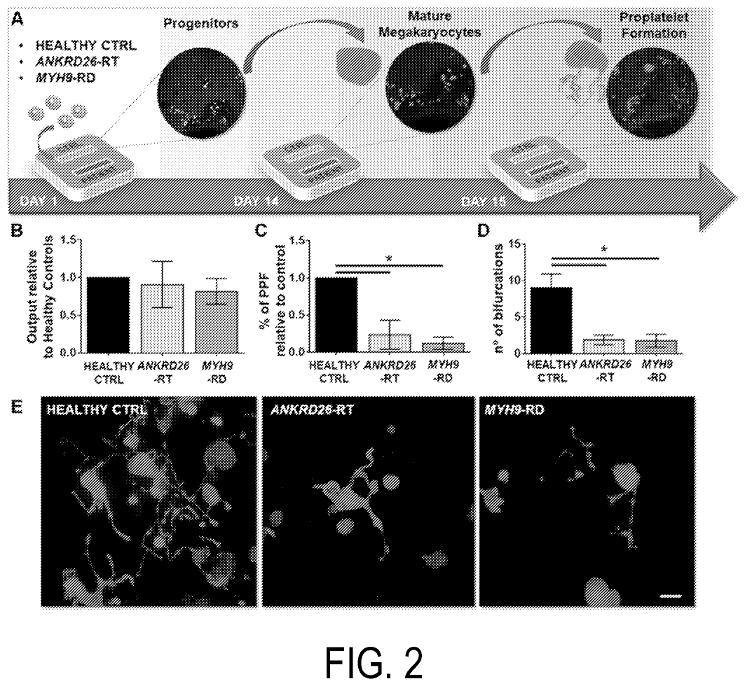

To ascertain the ability of the device to model physiologic and pathologic bone marrow, we took advantage of our expertise in culturing human hematopoietic stem cells (Currao, M., Malara, A., Di Buduo, C. A., Abbonante, V., Tozzi, L., & Balduini, A. (2015). Hyaluronan based hydrogels provide an improved model to study megakaryocyte-matrix interactions. Exp Cell Res; Di Buduo, C. A., Currao, M., Pecci, A., Kaplan, D. L., Balduini, C. L., & Balduini, A. (2016). Revealing Eltrombopag's promotion of human megakaryopoiesis through AKT/ERK-dependent pathway activation. Haematologica; Di Buduo, C. A., Wray, L. S., Tozzi, L., Malara, A., Chen, Y., Ghezzi, C. E., Smoot, D., Sfara, C., Antonelli, A., Spedden, E., Bruni, G., Staii, C., De Marco, L., Magnani, M., Kaplan, D. L., & Balduini, A. (2015a). Programmable 3D silk bone marrow niche for platelet generation ex vivo and modeling of megakaryopoiesis pathologies. Blood) and selected hematopoietic progenitors from peripheral blood of healthy controls and patients affected by two forms of Inherited Thrombocytopenia: ANKRD26-RT and MYH9-RD. The bone marrow device was able to support efficient differentiation of mature megakaryocytes from both healthy controls and patients (FIGS. 2A and 2B). However, patient-derived megakaryocytes displayed a decreased percentage of proplatelet formation by about 80%, accompanied by less branching of proplatelet shafts due to a significantly lower number of bifurcations as compared to healthy controls (FIG. 2C-E).

In order to validate the predictive value of the bone marrow response to drugs specifically targeting hematopoiesis, we chose Eltrombopag as the model compound since Eltrombopag represents to date the only tested drug shown to increase platelet count of patients with Inherited Thrombocytopenias. We tested the sensitivity of pathologic samples specifically in regard to megakaryocyte differentiation and platelet production among 24 cultures using samples from ANKRD26-RT and MYH9-RD patients. This cohort included 11 patients previously treated with Eltrombopag in a recent phase 2 clinical trial (Zaninetti, Gresele, et al., 2019) and 2 patients previously treated in preparation for elective surgery (Zaninetti, Barozzi, et al., 2019).

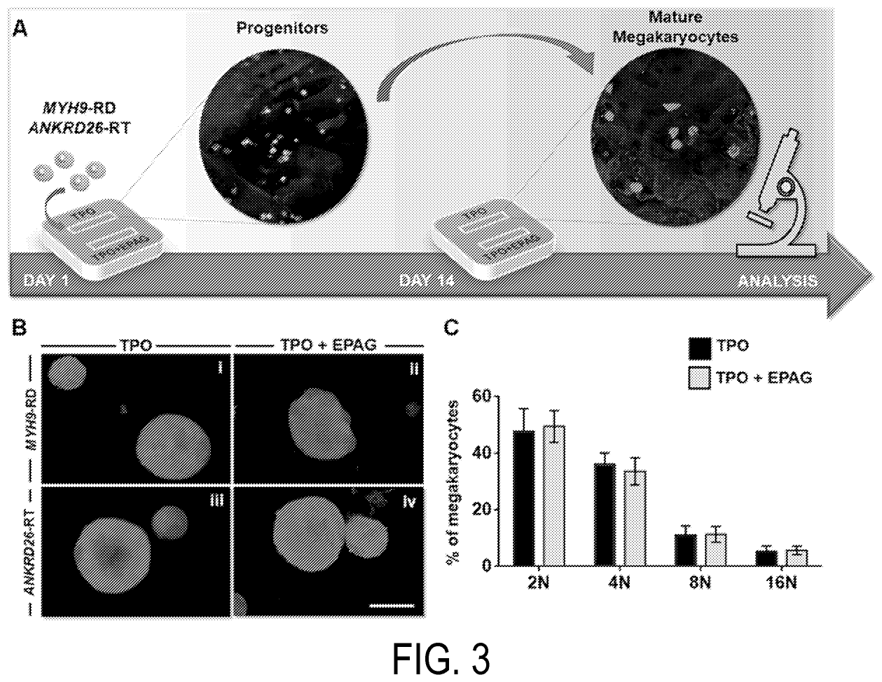

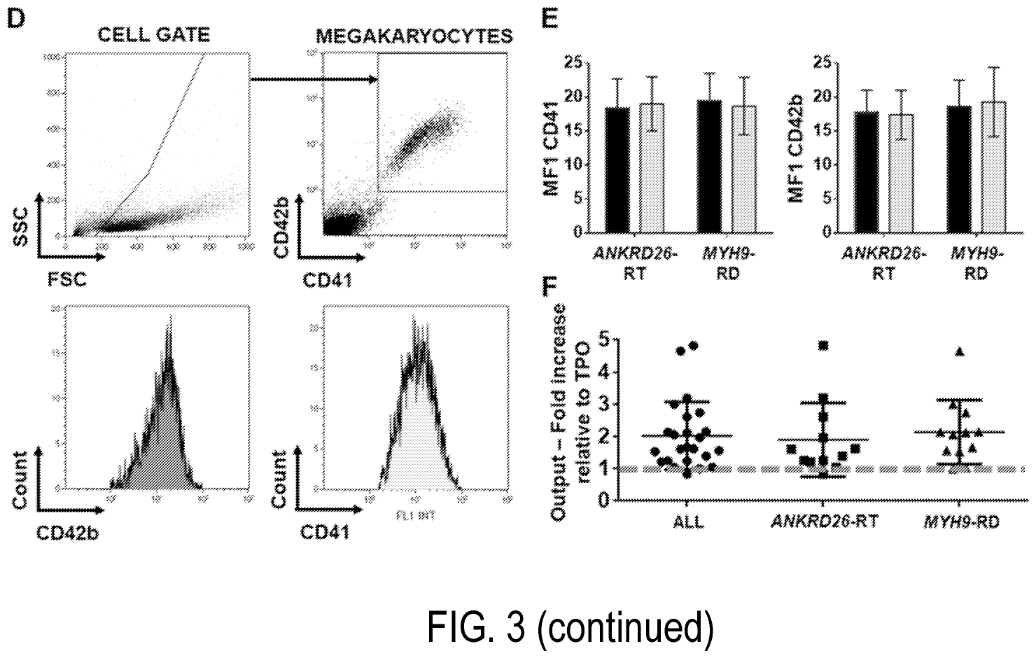

Blood samples for this study were collected when patients were out of Eltrombopag therapy and had platelet count at their baseline levels. Equal numbers of megakaryocytic progenitors were divided between each channel of the device and both were cultured in the presence of 10 ng/mL recombinant human TPO. One of the two channels was supplemented with 500 ng/mL Eltrombopag (FIG. 3A).

Insights into efficacy of Eltrombopag effects ex vivo were gained by simultaneously analyzing megakaryocyte differentiation at day 14 for each disorder. Specifically, cells were washed out of the device and analyzed. We observed comparable megakaryocyte maturation in terms of cell size (FIG. 3B), ploidy profile (FIG. 3C) and expression of lineage-specific markers (FIGS. 3D and 3E), with and without Eltrombopag. However, combination of TPO and Eltrombopag resulted in a significant two-fold increase in the output of mature megakaryocytes with respect to TPO alone for both ANKRD26-RT and MYH9-RD patients (FIG. 3F).

Assessment of Proplatelet Formation to Inform on Mechanisms of Action

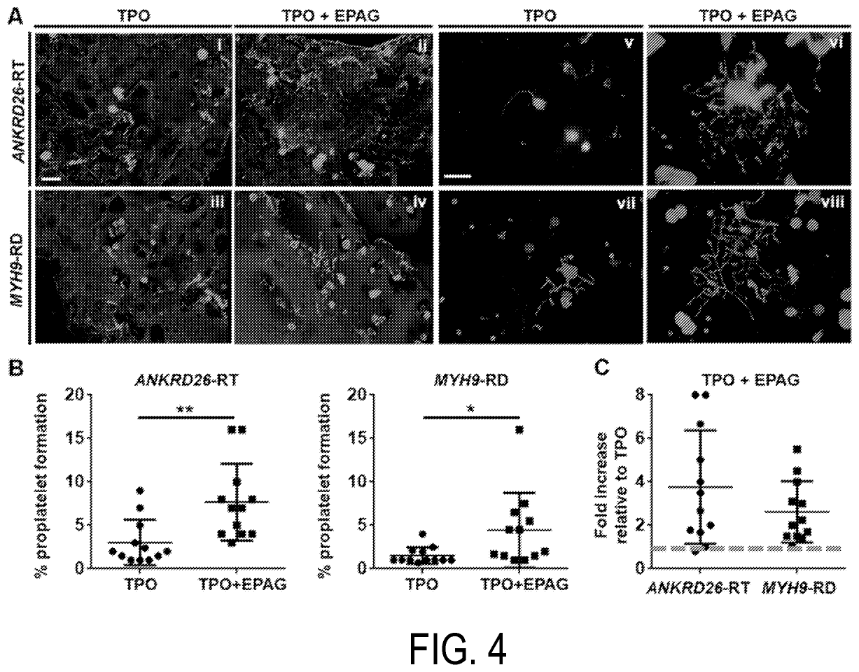

Confocal microscopy analysis of 3D scaffolds revealed a homogeneous distribution of CD61⁺megakaryocytes throughout the entire construct in both culture conditions, with more clusters in the presence of Eltrombopag, from both ANKRD26-RT and MYH9-RD (FIG. 4Ai-iv). Further, in the presence of Eltrombopag, megakaryocytes underwent characteristic cytoplasmic rearrangements typical of proplatelets (FIG. 4Aii,iv). β1-tubulin staining of megakaryocytes harvested from the device and seeded onto fibronectin-coated coverslips consistently highlighted that TPO in combination with Eltrombopag supported extension of multiple branched shafts resembling nascent platelets at their terminal ends (FIG. 4Av-viii) and a significant increase in the percentage of proplatelet-forming megakaryocytes in both ANKRD26-RT (FIGS. 4B and 4C).

### Ex Vivo Platelet Count as a Predictor of Drug Efficacy

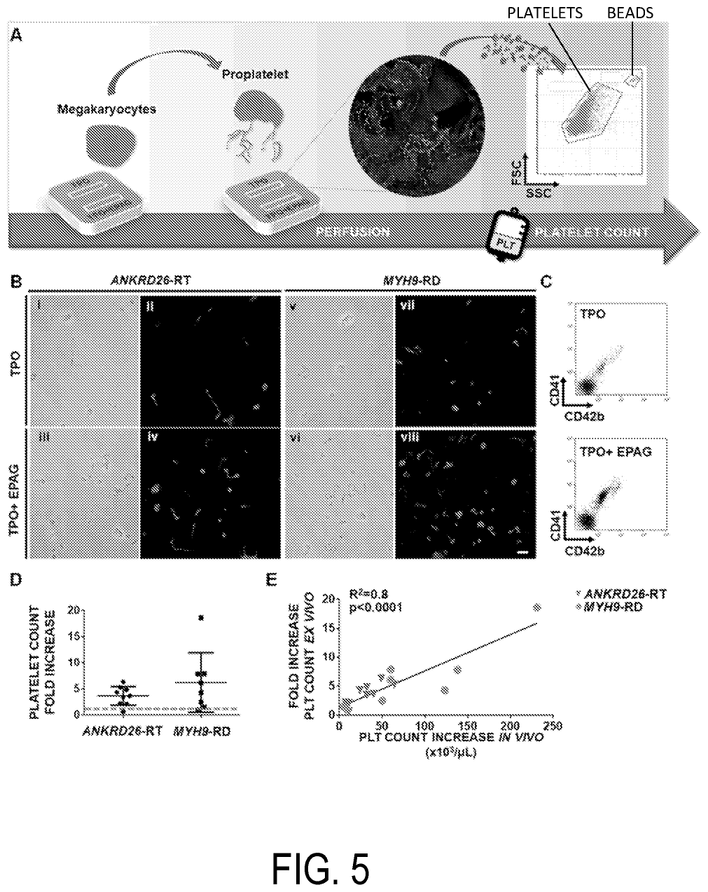

Since the desired ultimate effect of Eltrombopag in patients with ANKRD26-RT or MYH9-RD is an increase of platelet count, platelet production was the most pertinent parameter evaluated in our ex vivo model. Thus, we tested whether our device predicted the patient-specific response to Eltrombopag by performing a systematic study comparing extent of platelet production ex vivo with in vivo platelet response observed in the same patients (Zaninetti, Barozzi, et al., 2019; Zaninetti, Gresele, et al., 2019). At day 15 of culture, each channel of the device was connected to a peristaltic pump at the inlet and a gas-permeable collection bag at the outlet. The number of ex vivo produced platelets was evaluated by flow cytometry after 4 hours of perfusion at 37° C. and 5% CO₂ (FIG. 5A). Ex vivo collected platelets exhibited the β1-tubulin coil at their periphery, typically present in physiologic peripheral blood platelets (FIG. 5B), further supporting the physiological relevance of the reproduced bone marrow environment for replicating in vivo thrombopoiesis. Ex vivo collected platelets were double-stained with anti-CD41 and anti-CD42b antibodies and counted by flow cytometry with a bead standard (FIG. 5C). The number of CD41⁺CD42b⁺platelets collected per single channel was globally increased under treatment with TPO in combination with Eltrombopag with respect to TPO alone, in both ANKRD26-RT and MYH9-RD groups (FIG. 5D). However, platelet response was variable, with some samples demonstrating slight or no increase in ex vivo platelet production (FIG. 5D). When the fold increase of ex vivo response to Eltrombopag was compared with the increase in platelet count obtained in vivo after Eltrombopag administration in the same patients, there was a statistically significant correlation (R square=0.8; p<0.0001) (FIG. 5E). Accordingly, when the absolute numbers of platelet count increase ex vivo were compared to the response obtained in vivo there was a statistically significant correlation (R square=0.7; p<0.0001).

### DISCUSSION

Allogeneic platelet transfusions are widely used to treat acute bleeding in patients with thrombocytopenia of any origin and are also used to prevent bleeding in subjects who developed short-lasting, severe thrombocytopenia after chemotherapy or in those patients with more chronic thrombocytopenia in need of a procedure. However, platelet concentrates are not indicated for the prevention of hemorrhages in chronically thrombocytopenic patients for many reasons: they lose efficacy due to alloimmunization, acute reactions may occur, and transmission of infectious diseases is possible. Thus, platelet transfusions are not chronically administered to patients with inherited thrombocytopenia unless their platelet count is extremely low and their risk of bleeding is relatively high. Clearly, there is a need for alternative agents or approaches that could increase platelet count in these and chronic thrombocytopenic conditions.

Thrombopoietin-receptor agonists stimulate megakaryopoiesis and platelet production. Eltrombopag and/or Romiplostim and/or Avatrombopag are currently approved for treatment of primary immune thrombocytopenia at various stages of ITP in adults and children (Bussel, 2009), thrombocytopenia related to liver disease if a procedure is needed, and severe acquired aplastic anemia (Olnes et al., 2012). Small clinical trials and case reports have suggested that Thrombopoietin receptor agonists are also effective in increasing platelet counts in patients with certain forms of Inherited Thrombocytopenia (Rodeghiero et al., 2018) and that at least Eltrombopag could be used to replace platelet transfusions to prepare patients to undergo hemostatic challenges (Zaninetti, Barozzi, et al., 2019). Indeed, a few patients have successfully received long-term treatment with Thrombopoietin-receptor agonists, potentially paving the way for chronic treatment of these previously untreated forms of thrombocytopenia. However, platelet response to these drugs was variable among different patients, and sometimes the drugs were ineffective (Gerrits, A. J., Leven, E. A., Frelinger, A. L., Brigstocke, S. L., Berny-Lang, M. A., Mitchell, W. B., Revel-Vilk, S., Tamary, H., Carmichael, S. L., Barnard, M. R., Michelson, A. D., & Bussel, J. B. (2015). Effects of Eltrombopag on platelet count and platelet activation in Wiskott-Aldrich syndrome/X-linked thrombocytopenia. Blood, 126(11), 1367-1378; Zaninetti, Barozzi, et al., 2019; Zaninetti, Gresele, et al., 2019).

Here we have developed a 3D bone marrow tissue model that ex vivo reproduces in vivo platelet biogenesis in such a way that it allows us to predict response to Eltrombopag on a single patient basis. As proof of principle, we applied our system to study thrombocytopenic patients affected by ANKRD26-RT and MYH9-RD who were treated with the Thrombopoietin-receptor agonist Eltrombopag (Zaninetti, Gresele, et al., 2019). The results clearly demonstrate that this ex vivo tissue model can efficiently predict response to Eltrombopag in individual patients and allow more personalized patient treatment in the future reducing the number of non-responders unnecessarily exposed to potential side effects of treatment and to ineffective preparation for procedures. In the future, patients might be able to create their own platelets and thus avoid most if not all of the complications discussed above.

Inherited thrombocytopenias each represent a prototype of thrombocytopenias deriving from defective platelet biogenesis within the bone marrow. For many inherited thrombocytopenias, the mechanisms of defective platelet production remain unknown. Understanding the cause of thrombocytopenia in these diseases could define the most suitable treatment for each disorder and identify both novel potential targets and either novel drugs or novel uses of existing drugs. Current 2D assays for functional assessment of megakaryocytes do not effectively monitor the final stage of maturation, in particular proplatelet spreading, platelet formation, and platelet release (Balduini, A., Di Buduo, C. A., & Kaplan, D. L. (2016). Translational approaches to functional platelet production ex vivo. Thromb Haemost, 115(2), 250-256). By recreating megakaryocyte maturation from stem cells to platelet release, our miniaturized 3D bone marrow model demonstrated the ability to reproduce these key steps of thrombopoiesis, including alterations observed in diseased states.

Patient-derived iPSCs represent a potentially unlimited source of megakaryocytes that could be used to systematically study disease mechanisms and test candidate drug treatments. We hypothesize that combining the 3D bone marrow tissue model and iPSC technologies will be instrumental in addressing critical clinical needs for more specific understanding of the mechanism of action of Thrombopoietin-receptor agonists in patients.

Besides its ability to stimulate megakaryopoiesis, Eltrombopag has also been well-demonstrated to promote multilineage hematopoiesis in patients with acquired bone marrow failure syndromes. Although the exact mechanisms of its effects on hematopoietic progenitor cells are not completely clear, Kao et al. recently demonstrated a stimulatory effect on stem cell self-renewal independently of the Thrombopoietin receptor mediated through iron chelation-dependent molecular reprogramming. Our benchmark tests highlight that, beside platelet release, the 3D tissue model allowed us to track the effect of Eltrombopag on both progenitor cell and megakaryocyte functions, promising to provide a more comprehensive approach to study the effect of Thrombopoietin-receptor agonists on hematopoietic stem cells.

In summary, we developed a proof-of-concept system that in two weeks measures the impact of Thrombopoietin-receptor agonists on megakaryopoiesis and platelet production of individual patients starting from a small amount of their peripheral blood (FIG. 12). This silk-based technology, that can be produced and customized in 2 days, reaches the expectation of cost efficiency, time-saving, convenience, and personalization of modern therapeutic approaches. The data demonstrated that the ex vivo system could predict in vivo clinical response to Eltrombopag. The increase in the number of platelets collected in the ex vivo model was comparable to the increase of platelet count in vivo upon treatment with Eltrombopag. The broader impact of this work is in the design of tools to mimic the bone marrow ex vivo that can uncover mechanisms of impaired platelet production and enable testing of candidate drug treatments on platelet production using patient-derived cells. In the future, our system may serve as the basis for highly integrated approaches that could generate solutions for the ex vivo production of all blood cells for transfusion and therapeutic applications.

Each of the references cited herein are hereby incorporated by reference for all purposes.

Although this disclosure has described and illustrated certain embodiments, it is to be understood that the disclosure is not restricted to those particular embodiments. Rather, the disclosure includes all embodiments that are functional and/or equivalents of the specific embodiments and features that have been described and illustrated, and encompass various alternatives, modifications, and equivalents, as will be appreciated by those of skill in the art. Moreover, the features of the particular examples and embodiments, may be used in any combination. The present invention therefore includes variations from the various examples and embodiments, described herein, as will be apparent to one of skill in the art.

We claim:

1. A patient-specific method of administering a medicament to a patient in need thereof, wherein the medicament is indicated to modulate megakaryocyte differentiation, proplatelet formation, and/or platelet production in the patient, the method comprising:
a) preparing from cells acquired from the patient and/or acquiring from the patient patient-specific haematopoietic stem cells, patient-specific haematopoietic progenitor cells, patient-specific Induced Pluripotent Stem Cells, and/or patient-specific megakaryocyte progenitors;
b) seeding the patient-specific haematopoietic stem cells, the patient-specific haematopoietic progenitor cells, the patient-specific Induced Pluripotent Stem Cells, and/or the patient-specific megakaryocyte progenitors into at least two three-dimensional bone marrow models, each of the at least two three-dimensional bone marrow models comprising a regenerated silk fibroin sponge having an interconnected network of pores with pore sizes and pore distributions adapted to facilitate cell adhesion, proplatelet extension, proplatelet formation, and platelet release and collection from maturing haematopoietic stem cells, haematopoietic progenitor cells, Induced Pluripotent Stem Cells, and/or megakaryocyte progenitors seeded therein, the at least two three-dimensional bone marrow models including an experimental model and a control model, each of the regenerated silk fibroin sponges comprising at least one protein of the extracellular matrix;
c) introducing the medicament to the patient-specific hematopoietic stem cells, the patient-specific haematopoietic progenitor cells, the patient-specific Induced Pluripotent Stem Cells, and/or the patient-specific megakaryocyte progenitors within the experimental model;
d) perfusing the at least two three-dimensional bone marrow models with a cell culture medium;
e) maturing the patient-specific hematopoietic stem cells, the patient-specific haematopoietic progenitor cells, the patient-specific Induced Pluripotent Stem Cells, and/or the patient-specific megakaryocyte progenitors into patient-specific megakaryocytes, including cell adhesion, proplatelet extension, proplatelet formation, and platelet release and collection;
f) comparing platelet production from the patient-specific megakaryocytes within the experimental model versus the patient-specific megakaryocytes within the control model, the control model lacking the medicament;
g) generating a report, wherein the report includes a prediction of in vivo platelet production of the patient upon having the medicament administered, the prediction incorporating the comparing of step f), wherein the prediction is validated to have a statistically significant correlation between an increase in platelet count ex vivo and in vivo, expressed either as fold increase or absolute platelet numbers, with an R-squared value of at least 0.6 and a p value of less than 0.001 in a population study having at least 8 patients; and
h) subsequent to step g), administering medicament to the patient if the prediction exceeds a predetermined threshold and foregoing administering the medicament to the patient if the prediction fails to exceed the predetermined threshold.

2. The method of claim 1, wherein the at least two three-dimensional bone marrow models are substantially free of tubular blood vessel mimicking structures.

3. The method of claim 1 or 2, wherein the medicament is a Thrombopoietin-receptor agonist.

4. The method of any one of the preceding claims, wherein the medicament is Eltrombopag, Romiplostim, or Avatrombopag.

5. The method of any one of the preceding claims, wherein the medicament is Eltrombopag.

6. The method of any one of the preceding claims, wherein the patient has an ANKRD26-RT or MYH9-RD gene variant.

7. The method of any one of the preceding claims, the method further comprising analyzing megakaryocyte differentiation of the patient-specific megakaryocytes.

8. The method of the immediately preceding claim, wherein the report includes the megakaryocyte differentiation.

9. The method of any one of the preceding claims, wherein the interconnected network of pores comprises substantially uniform pores.

10. The method of any one of the preceding claims, wherein the at least one protein of the extracellular matrix is selected from the group consisting of a proteoglycan, hyaluronic acid, a collagen, elastin, fibronectin, fibrin, fibrinogen, a laminin, thrombospondin, and combinations thereof.

11. The method of any one of the preceding claims, wherein the at least one protein of the extracellular matrix comprises fibronectin.

12. A patient-specific method of predicting in vivo efficacy of a Thrombopoietin-receptor agonist for a patient, the method comprising:
a) preparing from cells acquired from the patient and/or acquiring from the patient patient-specific haematopoietic stem cells, patient-specific haematopoietic progenitor cells, patient-specific Induced Pluripotent Stem Cells, and/or patient-specific megakaryocyte progenitors;
b) seeding the patient-specific haematopoietic stem cells, the patient-specific haematopoietic progenitor cells, the patient-specific Induced Pluripotent Stem Cells, and/or the patient-specific megakaryocyte progenitors into at least two three-dimensional bone marrow models, each of the at least two three-dimensional bone marrow models comprising a regenerated silk fibroin sponge having an interconnected network of pores with pore sizes and pore distributions adapted to facilitate cell adhesion, proplatelet extension, proplatelet formation, and platelet release and collection from maturing hematopoietic stem cells, haematopoietic progenitor cells, Induced Pluripotent Stem Cells, and/or megakaryocyte progenitors seeded therein, the at least two three-dimensional bone marrow models including an experimental model and a control model, each of the regenerated silk fibroin sponges comprising at least one protein of the extracellular matrix;
c) introducing the medicament to the patient-specific hematopoietic stem cells, the patient-specific haematopoietic progenitor cells, the patient-specific Induced Pluripotent Stem Cells, and/or the patient-specific megakaryocyte progenitors within the experimental model;
d) perfusing the at least two three-dimensional bone marrow models with a cell culture medium;
e) maturing the patient-specific haematopoietic stem cells, the patient-specific haematopoietic progenitor cells, the patient-specific Induced Pluripotent Stem Cells, and/or the patient-specific megakaryocyte progenitors into patient-specific megakaryocytes, including cell adhesion, proplatelet extension, proplatelet formation, and platelet release and collection;
f) comparing platelet production from the patient-specific megakaryocytes within the experimental model versus the patient-specific megakaryocytes within the control model, the control model lacking the medicament; and
g) generating a report, wherein the report includes a prediction of in vivo platelet production of the patient upon having the medicament administered, the prediction incorporating the comparing of step f), wherein the prediction is validated to have a statistically significant correlation between an increase in platelet count ex vivo and in vivo, expressed either as fold increase or absolute platelet numbers, with an R-squared value of at least 0.6 and a p value of less than 0.001 in a population study having at least 8 patients.

13. The method of claim 12, wherein the at least two three-dimensional bone marrow models are substantially free of tubular blood vessel mimicking structures.

14. The method of claim 12 or 13, wherein the medicament is a Thrombopoietin-receptor agonist.

15. The method of any one of claim 12 to the immediately preceding claim, wherein the medicament is Eltrombopag, Romiplostim, or Avatrombopag.

16. The method of any one of claim 12 to the immediately preceding claim, wherein the Thrombopoietin-receptor agonist is Eltrombopag.

17. The method of any one of claim 12 to the immediately preceding claim, wherein the patient has an ANKRD26-RT or MYH9-RD gene variant.

18. The method of any one of claim 12 to the immediately preceding claim, the method further comprising analyzing megakaryocyte differentiation of the patient-specific megakaryocytes.

19. The method of any one of claim 12 to the immediately preceding claim, wherein the report includes the megakaryocyte differentiation.

20. The method of any one of claim 12 to the immediately preceding claim, wherein the interconnected network of pores comprises substantially uniform pores.

21. The method of any one of claim 12 to the immediately preceding claim, wherein the at least one protein of the extracellular matrix comprises a proteoglycan, hyaluronic acid, a collagen, elastin, fibronectin, fibrin, fibrinogen, a laminin, thrombospondin, or a combination thereof.

22. The method of any one of claim 12 to the immediately preceding claim, wherein the at least one protein of the extracellular matrix comprises fibronectin.

23. A kit comprising the three-dimensional bone marrow model of the method of any one of the preceding claims and instructions describing the method of any one of the preceding claims.
